(19) United States
(12) Patent Application Publication
(10) Pub. No.: US 2003/0162957 A1
(43) Pub. Date: Aug. 28, 2003

(54) Protected deoxyadenosines and deoxyguanosines

(76) Inventors: Charles C. Wu, Denville, NJ (US); Jing-Ji Ma, West Seneca, NY (US); Stephen F. Yates, Arlington Heights, IL (US)

Correspondence Address: Synnestvedt & Lechner LLP, 2600 Aramark Tower, 1101 Market Street, Philadelphia, PA, 19107-2950, US

(21) Appl. No.: 10/083,216

(22) Filed: Feb. 26, 2002

(51) Int. Cl. C07H 19/167; C07H 19/173

(52) U.S. Cl. 536/27.3; 536/27.81

(57) ABSTRACT

Processes are disclosed for the preparation of either an N-acyl deoxyadenosine or an N-acyl deoxyguanosine by acylating the hydroxyl groups and the exocyclic amino group of a corresponding deoxyadenosine or deoxyguanosine with anhydride to form a 3′-, 5′-O-acyl, N-acyl deoxyneucloside and then selectively removing the acyl groups from the hydroxyl groups to form an N-acyl deoxyadenosine or N-acyl deoxyguanosine.

### FIELD OF THE INVENTION

The invention relates to the synthesis and purification of protected nucleosides, and more particularly to methods for the synthesis and purification of protected nucleosides without the use of pyridine as a solvent.

Nucleosides are compounds of importance in physiological and medical research, obtained during partial decomposition, i.e., hydrolysis, of nucleic acids, and containing a purine or pyrimidine base linked to either D-ribose (forming ribonucleosides) or D-deoxyribose (forming deoxyribonucleosides). They are nucleotides minus the phosphate group. Well-known nucleosides include adenosine (A), cytidine (C), uridine (U), and guanosine (G), as well as the deoxynucleosides deoxyadenosine (dA), deoxycytidine (dC), deoxyguanosine (dG), and deoxythymidine (dT). It should be noted that thymidine is actually a deoxynucleoside, and may be referred to in the literature as either thymidine (T) or deoxythymidine (dT).

The “deoxy” site in each of the four deoxynucleosides dA, dC, dG and dT compound is at the 2′ position of the furan ring. The active hydroxy sites are at the 3′ and 5′ positions. In each of dA, dC and dG there is an exocyclic NH₂ group which is protected, preferably by acylation, as discussed below.

Nucleosides are multi-functional compounds, having both amino and hydroxy functional groups. They may be used in automatic synthesizers to produce oligonucleotides as well as synthetic genes. Oligonucleotides and genes are formed by stringing together nucleosides in a predetermined sequence through phosphate ester linkages between the 3′-hydroxyl group of one nucleoside and the 5′-hydroxyl group of the next.

In order to conduct syntheses selectively and efficiently, it is necessary to block or “protect” specific functional groups in order to achieve reaction at the desired sites. The “protecting” groups are designed to be removed under specific carefully controlled conditions, usually under relatively mild and typically acidic conditions. To be useful as precursors in the synthesis of high value pharmaceuticals, it is necessary that protected nucleosides be of very high purity (i.e., greater than about 99%, preferably greater than about 99.5%) Unless otherwise indicated, purity percentages herein are expressed as percent area as measured by HPLC, but may be expressed as percent by weight, where indicated.

### Reported Developments

Typically, the protection of nucleosides involves the derivatization of both amino and hydroxy functional groups, except for thymidine, which requires only the protection of hydroxyl groups. Various schemes are employed to achieve these protected nucleosides, but usually the N-protected derivatives (most often N-acylated) are isolated and purified before protecting the hydroxyl groups. For example, the 2-amino group of dC, and the 6-amino group of dA are protected by benzoyl groups, while the 2-amino group of dG is protected by an isobutyryl group. The hydroxyl group, typically a 5′-hydroxyl group in all of the nucleosides, is generally protected by a 4,4′-dimethoxytrityl (DMT) group.

When phosphorylated, a protected nucleoside will react at the 3′-hydroxyl group. A phosphorylated protected nucleoside will then react with a second nucleoside at the 5′ position after the 5′ position has been deblocked by removal of the DMT group. The phosphorylation thus occurs between the 3′-hydroxyl group of the first nucleoside and the 5′-hydroxyl group of the second nucleoside to form a dinucleotide. By repeating the phosphorylation procedure, the synthesizer can produce oligonucleotides containing a predetermined sequence of nucleosides.

Discussions of the synthesis and protection of nucleosides by derivatization may be found in many references, including the following, all of which are incorporated herein by reference. One method of protecting nucleosides is described in Ti, et al., “Transient Protection: Efficient One-flask Syntheses of Protected Deoxynucleosides”, J. Am. Chem. Soc., Vol. 104, 1316-1319 (1982), which is discussed in more detail below in regard to the examples. Other methods of synthesizing protected nucleosides are set forth in Charubala, et al., “Nucleotides XXIII: Synthesis of Protected 2′-Deoxyribonucleoside-3′-phosphotriesters Containing the p-Nitrophenylethyl Phosphate Blocking Group”, Synthesis, 965 (1984). Still other methods for synthesizing such protected nucleosides are set forth in Kierzek, “The Synthesis of 5′-O-dimethoxytrityl-N-acyl-2′-deoxynucleosides, Improved ‘Transient Protection’ Approach”, Nucleosides & Nucleotides, 4(5), 641-649 (1985). In all of these references, protection by N-acylation is effected with benzoyl chloride on adenosine and cytidine derivatives, and with isobutyric anhydride on guanosine derivatives, as is well known in the art. The compounds are then further protected by the introduction of methoxytrityl or dimethoxytrityl groups, also as is well known in the art. An earlier article on the protection of such nucleosides may be found in Schaller, et al., J. Amer. Chem. Soc., Vol. 85, 3821-3827 (1963). Another article on protected nucleosides is McGee, et al., “A Simple High Yield Synthesis of N²-(2-Methylpropanoyl)-2′-deoxyguanosine”, Synthesis, 540 (1983). In all of the reported syntheses, the protected nucleosides must be subjected to purification prior to their use in pharmaceutical syntheses.

A detailed description of the preparation of protected deoxynucleosides is provided in Jones, “Preparation of Protected Deoxyribonucleosides”, Oligonucleotide Synthesis: A Practical Approach, IRL Press, 23-34 (1984), incorporated herein by reference. This reference describes various processes for protecting such nucleosides, protecting the 5′-hydroxyl group as a trityl ether and the 3′-hydroxyl group with benzoyl chloride or a levulinic anhydride. For ribonucleosides, protection of the 2′-hydroxyl group is also needed, most commonly provided by tert-butyldimethylsilyl (TBDMS) group. The exocyclic amino groups are protected by acylation with a benzoyl moiety (Bz) or an isobutyryl group (iB), as discussed above. Acyl protection is also discussed in Köster, et al., “N-Acyl Protecting Groups for Deoxynucleosides”, Tetrahedron, 37(2), 363-369 (1981). TBDMS is discussed for protection of any hydroxyl group, using 4-(dimethylamino)pyridine (DMAP) as a catalyst, in Chaudhary, et al., “4-Dimethylaminopyridine: An Efficient and Selective Catalyst for the Silylation of Alcohols”, Tetrahedron Letters, No. 2, 99-102 (1979).

All of these reported preparation procedures use pyridine as a solvent in the synthesis processes. Pyridine has been found to be chemically compatible with the nucleosides, the protected nucleosides, and with the various reactants used to form such products. However, pyridine is highly toxic and its ability to dissolve nucleosides is limited. Large amounts of pyridine have to be used in the reaction to maintain the nucleosides in solution. These large amounts of pyridine are difficult to remove from the protected nucleoside products. Thus, the pyridine solvent processes have very low productivity, and the processing costs are high. It would, therefore, be desirable to have a process for preparing protected nucleosides that does not require the use of pyridine as a solvent.

As discussed in Chaudhary, et al., cited above, 4-(dimethylamino)pyridine (DMAP) is often used as a catalyst in processes in which pyridine is the solvent. In fact, DMAP has become a standard catalyst for use in such processes. However, DMAP is also considered highly toxic, is very expensive, and is difficult to separate from the desired product. It is, therefore, also desirable to provide a process which does not require the use of DMAP as a catalyst.

Another reason for the use of pyridine is that it is a mild base, and can neutralize acids formed in the reaction mixture. In this capacity it acts as an “acid scavenger”. Many of the chemical reactions in the protection process produce acid byproducts, such as HCl. For example, benzoyl chloride or isobutyryl chloride are used to acylate the amino group. In the acylation reaction, these acid chloride compounds react with the amino group to produce HCl as a byproduct.

Similarly, in the tritylation reaction, dimethoxytrityl chloride (DMT-Cl) or other trityl chlorides react with hydroxy groups to generate HCl as a byproduct of this reaction. It is necessary to neutralize the acid produced by these reactions to prevent the acid from undesirably reacting with the nucleosides, and therefore an acid scavenger is needed in such processes.

The automatic synthesis of oligonucleotides as well as synthetic genes has previously been carried out on a milligram scale for research purposes. More recently, oligonucleotides are being used in larger quantities in the formation of commercial products such as anti-sense drugs that prevent the synthesis of disease-causing proteins in the human body. The very sensitive nature of the protecting groups together with the variety of polar and non-polar impurities generated during the syntheses of these derivatives makes their purifications complicated, expensive, and difficult to scale-up to industrial-scale production. Therefore, procedures are now needed to manufacture nucleosides in relatively large industrial quantities.

Column chromatography, especially flash silica gel chromatography, has been used extensively to purify protected nucleosides on a small to medium scale. This method requires the use of large volumes of high purity solvents in proportion to the amount of material purified. The method is also labor-intensive, requiring precise monitoring to make the fraction cuts at the appropriate times to maximize yield of desired product. For these reasons, large-scale use of this method of purification can be very costly.

The equipment required to conduct flash silica gel chromatography on a multi-kilogram scale is expensive to purchase and operate. For example, one commercially available production scale chromatography unit is capable of separating up to about 4 kg of material per run. Run times can vary from 18 to 36 minutes, at an elution rate of 7 liters per minute. The basic unit investment is very expensive, coupled with the cost (and subsequent disposal cost) of 125 to 250 liters of expensive high purity solvent per run. In addition, some products need to be purified multiple times by chromatography to reach the desired purity, each time at a significant yield loss. The high costs associated with chromatographic purification make it unattractive to industrial-scale operations.

For the above reasons, it is desirable to provide a process for protecting nucleosides which does not required the use of pyridine as a solvent, which does not require the use of DMAP as a catalyst, and which does not require the use of separative chromatography to purify the product. The present invention meets these needs and offers additional advantages as discussed in the following detailed description.

The process of acylation for protection of exocyclic amino groups is well known in the art and is described, for example, in Jones, “Preparation of Protected Deoxyribonucleosides,” op. cit. As discussed in that reference, a key problem in the preparation of protected deoxyribonucleosides is chemically differentiating between hydroxyl and amino groups. Only in the case of dC has it been possible to selectively acylate the amino group (N-acylation) without acylating the hydroxyl group. The dA and dG amino groups have been found to be too weakly basic for such a selective reaction. However, it is possible to selectively de-acylate, that is, to differentiate by making use of the more rapid hydrolysis, at pH greater than 10, of the esters versus the amides. Thus the chemical procedure which has been used is to per-acylate the nucleoside, acylating both the hydroxyl groups and the amino group, and then to selectively hydrolyze the esters to leave the desired N-acylated nucleoside. Such a per-acylation process, however, has been found to be difficult to use, because it requires isolation of the per-acylated intermediate. An alternative procedure is discussed in the Jones reference for selective N-acylation based on temporary protection of the hydroxyl groups as trimethylsilyl ethers, and is referred to as “TMS-transient protection.” Unlike ester groups, the trimethylsilyl ethers can be hydrolyzed in solution without the need for isolation. For these reasons, the TMS-transient protection method of N-acylation is the preferred method for use with dA and dG. However, the acylation procedures described in the Jones reference require the use of pyridine as a solvent.

In PCT Published application WO 0075154, the applicants disclosed a process for selectively protecting the exocyclic amino group of a starting deoxyribonucleoside which has an exocyclic amino group which is to be protected by acylation and at least one hydroxyl group which is to be left unprotected, the process comprising:

a) dispersing the starting deoxyribonucleoside in a polar solvent which is substantially free of pyridine and which is a solvent for the protected product formed in step b); and

b) selectively acylating said exocyclic amino group of said starting deoxyribonucleoside to form a protected product in which said hydroxyl group(s) are unprotected.

This method for the N-acylation of deoxyribonucleosides does not use pyridine as a solvent or DMAP as a catalyst and eliminates the need for the difficult step of separating product from pyridine solvent and/or DMAP catalyst, as generally required in prior processes. This process provides also protected nucleosides that may be purified by liquid-liquid extraction and/or selective adsorption, and then solidified by precipitation or crystallization by adding a solvent in which the product is insoluble. The PCT publication discloses that the pyridine-free synthetic method varies for each of the three deoxyribonucleosides that require acylation, dA, dC and dG. The method requires that dA and dG be acylated indirectly via TMS-transient protection while dC can be acylated directly by selective acylation.

Applicants have improved upon the pyridine-free synthetic method as it relates to the dA and dG thereby simplifying and improving the overall yield of the protection method for the synthesis of protected dA and dG.

### SUMMARY OF THE INVENTION

The present invention relates to a process for the preparation of an N-acyl deoxynucleoside, which is either an N-acyl deoxyadenosine or an N-acyl deoxyguanosine, comprising acylating the hydroxyl groups and the exocyclic amino group on said deoxynucleoside with anhydride to form a 3′-, 5′-O-acyl, N-acyl deoxynucleoside and selectively removing the acyl groups from the hydroxyl groups to form an N-acyl deoxyadenosine or N-acyl deoxyguanosine.

Another aspect of the present invention relates to a process for the preparation of an N-acyl derivative of a deoxynucleoside containing 3′- and 5′-hydroxyl groups and an exocyclic amino group, comprising the steps of:

(1) reacting said deoxynucleoside with a first anhydride under conditions effective in selectively acylating said hydroxyl groups;

(2) reacting the 3′-, 5′-O-acylated product of step (1) with a second anhydride under conditions effective in acylating said primary amino group to form an N-acylated, 3′-, 5′O-acylated deoxynucleoside; and

(3) subjecting said N-acylated, O-acylated deoxynucleoside to conditions effective to selectively remove said O-acyl groups to form N-acyl deoxynucleoside,

wherein said deoxynucleoside is either deoxyadenosine or deoxyguanosine.

The present invention is further an improvement in a process for the protection of the exocyclic amino group and 5′-hydroxyl group contained in either deoxyadenosine or deoxyguanosine, comprising the transient protection of the 3′- and 5′-hydroxyl groups, the acylation of said amino group, the selective removal of said hydroxyl group protection and a non-transient protection of said 5′-hydroxyl group, wherein the improvement comprises the selective transient acylation of said deoxynucleoside hydroxyl groups, the acylation of the exocyclic amino group, and the selective removal of the O-acyl protecting groups.

The practice of the present invention is an unexpected improvement in the prior art pyridine-free nucleoside synthetic method providing for simplified processing and higher yields than available using the methods described in the prior art.

### BRIEF DESCRIPTION OF THE DRAWINGS

FIG. 1 presents a schematic representation of the preparation of protected N⁶-benzoyl-5′-O-(4,4-dimethoxytrityl)-2′-deoxyadenosine from deoxyadenosine free-base.

FIG. 2 presents a schematic representation of the preparation of protected 5′-O-dimethoxytrityl N²-isobutyryl-2′-deoxyguanosine from deoxyguanosine free-base.

### DETAILED DESCRIPTION

The process of the present invention is applicable to the preparation of protected deoxyribonucleosides, and is particularly pertinent to improving the methods used in the prior pyridine-free synthetic process of protected deoxynucleosides discussed above. In that process, the deoxynucleosides dA, dC and dG, each of which have an exocyclic amino (NH₂), are first protected by acylation of that amino group. This step is necessary, because the highly reactive amino groups would otherwise react with the compounds being used to protect the hydroxyl group. As discussed above, the amino groups of dA and dC are protected preferably by benzoyl groups, while the amino group of dG preferably is protected by an isobutyryl group. These acylated compounds will be referred to herein as Bz-dA, Bz-dC and iB-dG, respectively.

The preferred acylation procedures for protecting exocyclic amino groups vary for each of the three deoxyribonucleosides that require acylation, dA, dC and dG. The prior art disclosed that dC could be acylated directly by selective N-acylation, while dA and dG could not be so acylated and therefore preferably are acylated indirectly via TMS-transient protection. Applicants have discovered a process modification that enables the direct N-acylation of dA and dG in a pyridine free solvent system and that avoids the need for transient TMS protection.

The solvent chosen for the protective acylation reactions should be one in which the N-acylated deoxynucleoside produced is soluble. The starting deoxynucleoside need not be fully soluble in the solvent provided it can be dispersed to carry on the acylation process. The preferred polar solvent to use depends on the particular deoxynucleoside being acylated, as discussed further below.

The process according to the present invention achieves the N- and O-acylation using organic anhydride reagents. The use of anhydrides in the present method prevents the generation of strong acids during the acylation reaction, and as applicants have discovered, permits the choice of conditions for selective O-acylation of dG and dA, as well as simultaneous O- and N-acylation, if desired.

Preferred anhydride reagents comprise the alkyl anhydrides and the aryl anhydrides. More preferred anhydrides include the lower alkyl anhydrides and the phenyl anhydrides. Lower alkyl groups as described herein mean straight chain or branched alkyl groups containing one to about 6 carbon atoms. Exemplary lower alkyl groups include methyl, ethyl, n-propyl, isopropyl, n-butyl, isobutyl, n-pentyl, and n-hexyl. Preferred anhydrides include acetic anhydride, isobutyryl anhydride and benzoic anhydride.

The present process selectively removes the O-acyl protecting groups under conditions that leave the N-acyl group intact. Selective removal may be achieved by the use of a nucleophilic reagent in a non-aqueous polar protic solvent. A preferred nucleophilic reagent is a salt of a lower alkyl alkoxide, such as methoxide, ethoxide or n-propoxide. Metal salt alkoxides are preferred. The protic polar solvent typically corresponds to the choice of metal alkoxide, and is preferably methanol, ethanol or propanol, respectively. The de-protection reaction is typically conducted at low temperature to provide for selective hydroxyl de-protection. A preferred temperature range is from about −35° C. to about 0° C., more preferably from about −25° C. to about −10° C., and most preferably at about −15° C.

If the N-protecting group is a lower alkyl-protecting group, then the N-acylation step may be achieved in a non-selective first acylation, followed by a selective de-protection of the acylated hydroxyl groups. In this case, only one anhydride need be used in the practice of the present invention. This is the preferred method of preparing 5′-O-dimethoxytrityl N²-isobutyryl-2′-deoxyguanosine described in more detail below.

The direct acylation of dG will be discussed first. In this acylation procedure, a starting material of dG monohydrate preferably is used as the starting deoxynucleoside. The dG is dispersed in a polar aprotic solvent that is free of pyridine. The solvent is preferably as anhydrous as possible, because any water present must be removed or otherwise tied up prior to acylation. Suitable aprotic solvents include tetrahydrofuran (THF), acetonitrile, amides, such as dimethylformamide (DMF), dimethylacetamide and N-methylpyrrolidone, as well as dimethylsulfoxide (DMSO), dimethylsulfone and hexamethylphosphate (HMPA). Preferred polar aprotic solvents for use in the direct acylation of dG are aprotic solvents such as dimethylformamide (DMF). An acylating reagent, preferably an anhydride is added to the dG solution. Isobutyric anhydride is a suitable and preferred reagent. A preferred molar ratio of anhydride to dG is about 4 to 5. The reaction temperature should be maintained at about 70 to about 90 degrees C., and most preferably between about 75 to about 80 degrees C. The reaction is slower at lower temperatures, which may result in an incomplete reaction that may contaminate the product with unreacted starting materials. The reaction mixture may be monitored to ensure complete reaction of the starting material and conversion of all partially acylated intermediates to the final tri-acylated product. A number of monitoring means may be used including NMR, UV, IR and various forms of chromatography. The preferred monitoring means is high-pressure liquid chromatography (HPLC).

The anhydride acylating agent added to the starting deoxyribonucleoside solution produces organic acid byproducts upon reacting with the nucleosides. In such cases, the acid byproduct must be neutralized, or it may cause decomposition of the nucleoside. Therefore, an acid scavenger preferably is included in the reaction mixture. A preferred acid scavenger is a tertiary amine, which forms a salt with the acid, with a particularly preferred tertiary amine being triethylamine (TEA). Other suitable tertiary amines include any tri-alkyl amine of the general formula N(R)₃, wherein R represents the same or different C₁ to C₆ alkyl groups. In addition to TEA, such tri-alkyl amines include trimethylamine, tripropylamine and di-isopropyl-monoethyl amine (DIPEA). Other suitable tertiary amines include triethanolamine.

The acylation of dA is achieved by selective O-acylation using a lower alkyl anhydride, followed by N-benzoylation, and then selective deprotection of the hydroxyl sites to leave just the amino site benzoylated, thus forming benzoylated dA (Bz-dA). The solvent in which the starting dA is dispersed is preferably an aprotic solvent, preferably as anhydrous as possible. In a preferred embodiment of this process, dA monohydrate (dA·H₂O) is dissolved in toluene with about x equivalents of triethylamine (TEA). Of the x eq of TEA, 7 eq is to neutralize acetic acid produced from the chemical reactions and 3 eq to buffer the reaction mixture to ensure a slightly basic environment. About 2-4 equivalents of acetic anhydride is added slowly while maintaining the reaction at a relatively low temperature of about 15 to 30 degrees C., preferably about 20 to 25 degrees C. The acetic anhydride selectively reacts at the active OH and hydroxyl sites. The reaction should be monitored, as by TLC and/or HPLC, until all of the dA is consumed. The reaction is quenched with an aqueous mild base such as sodium bicarbonate to consume all unreacted anhydride.

The next step uses the organic reaction mixture containing the O-acylated compound without isolation to acylate the unreacted exocyclic amino group. The typical protecting group for dA is benzoyl, and is the preferred N-acyl protecting group in the present method, although other anhydride may also be used in the N-acyl reaction. The N-acylation conditions are harsher than the earlier O-acylation reaction in that the reaction temperature should be maintained at about 75 to about 95 degrees C., preferably between about 80 to about 90 degrees C., and most preferably between about 83 to about 88 degrees C. The reaction is slower at lower temperatures, which may result in an incomplete reaction that may contaminate the product with unreacted starting materials. To ensure completion of the reaction, the reaction mixture may be monitored to ensure complete reaction of the starting material and conversion of all partially acylated intermediates to the final N-benzoylated product. A number of monitoring means may be used including NMR, UV, IR and various forms of chromatography. The preferred monitoring means is high-pressure liquid chromatography (HPLC).

As in the first anhydride reaction, the anhydride produces organic acid byproducts, such as benzoic acid, upon reacting with the dA. In such cases, the acid byproduct must be neutralized, or it may cause decomposition of the nucleoside. Therefore, an acid scavenger preferably is included in the reaction mixture. A preferred acid scavenger is a tertiary amine, which forms a salt with the acid, with a particularly preferred tertiary amine being triethylamine (TEA). Other suitable tertiary amines include any tri-alkyl amine of the general formula N(R)₃, wherein R represents the same or different C₁ to C₆ alkyl groups. In addition to TEA, such tri-alkyl amines include trimethylamine, tripropylamine and di-isopropyl-monoethyl amine (DIPEA). Other suitable tertiary amines include triethanolamine.

The acetylated hydroxyl groups are then deprotected and restored to their original hydroxyl form by hydrolysis. The hydrolysis reaction selectively deprotects the hydroxyl sites, while leaving the amino sites benzoylated. A preferred hydrolysis method is achieved by first treating the protected dA with an alcoholic alkoxide, such as methanolic sodium methoxide to remove the acetyl groups and form the sodium salts of the N-benzoyl dA. Subsequent treatment with an aqueous buffered salt solution, preferably an aqueous solution of a tertiary amine acid addition salt, such as triethyl amine hydrochloride, converts the sodium hydroxyl salt of dG to the original hydroxyl groups. The deprotected N-benzoyl dA is separated in the organic phase of the reaction mixture, which phase is separated and dried for subsequent use to form the 5′-O-protected N-benzoyl dA.

The pyridine-free deoxyribonucleosides which have had their exocyclic amino groups protected by acylation in accordance with the present invention may now be used to form 5′-protected deoxyribonucleoside products as described in the prior pyridine-free process. In that process, the 5′-hydroxyl group of the deoxyribonucleoside is protected, and the protected product is purified and separated out of solution as a solid. Purification and solidification comprise liquid-liquid extraction steps that take advantage of the fact that the protected nucleoside products are insoluble in water, while soluble in selected polar and non-polar solvents.

There is therefore provided an improved process for preparing an essentially pure 5′-protected deoxyribonucleoside comprising:

a) dissolving a starting deoxyribonucleoside prepared in accordance with the present invention, in a polar, aprotic solvent which is inert to the starting deoxyribonucleoside, to the 5′-protected deoxyribonucleoside, and to the other reactants, wherein any exocyclic amino groups of the starting deoxyribonucleoside are protected, preferably by acyl protection;

b) reacting the starting deoxyribonucleoside in said solution with a protecting reagent to form a 5′-protected deoxyribonucleoside product;

c) removing polar impurities by one or more liquid-liquid extractions using immiscible polar and non-polar solvent systems in which the product preferentially partitions into the non-polar phase, and the impurities preferentially partition into the polar phase; and

d) removing non-polar impurities by solidifying the product out of solution while leaving the non-polar impurities in solution.

In step (a), the starting deoxyribonucleoside may be one that already has its exocyclic amino group protected. Protection of the exocyclic amino group may be made by the above-described acylation protection process of the present invention. The nucleoside to be protected is dissolved in a polar, aprotic organic solvent. Examples of polar, aprotic solvents suitable for use in this step include amides, such as dimethylformamide (DMF), dimethylacetamide and N-methyl-pyrrolidone, as well as dimethylsulfoxide (DMSO), dimethylsulfone and hexamethylphosphate (HMPA). DMF is particularly preferred. For the reasons discussed above, pyridine is not used as a solvent in the processes of the present invention.

In step (b), a protecting reagent is added to the starting deoxyribonucleoside solution that selectively protects the 5′-hydroxyl group of the deoxyribonucleoside. The protecting reagent must react with the 5′ hydroxyl group preferentially over the 3′ hydroxyl group to form a removable protecting group at the 5′ position. A preferred method of protecting this hydroxyl group is to form a trityl derivative compound, a process referred to as “tritylation”. Preferred protecting groups are trityl, methoxytrityl and dimethoxytrityl groups. Preferred protecting reagents are trityl chloride and substituted trityl chlorides. For example, dimethoxytrityl chloride (DMT-Cl) is used in the examples set forth below. During the tritylation reaction, the 3′-hydroxyl group should be left unreacted. This is necessary to permit selective bonding of the 5′ and 3′ positions during subsequent oligonucleotide synthesis. The tritylation reaction is preferably conducted at a temperature ranging from about −10° C. to about 40° C., more preferably from about 10° C. to about 25° C.

Protecting reagents such as trityl chloride or substituted trityl chlorides produce an acid byproduct upon reacting with nucleosides. In such cases, the acid byproduct must be neutralized, or it may cause decomposition of the nucleoside. Therefore, an acid scavenger is preferably included in the reaction mixture. Preferably the acid scavenger is present in a mole ratio to the tritylation agent of about 1:1 to about 3:1. A preferred acid scavenger is a tertiary amine, which forms a salt with the acid. A particularly preferred tertiary amine is triethylamine (TEA). Other suitable tertiary amines include any tri-alkyl amine of the general formula N(R)₃, wherein R represents the same or different C₁ to C₆ alkyl groups. In addition-to TEA, such tri-alkyl amines include trimethylamine, tripropylamine and di-isopropyl-monoethyl amine (DIPEA). Other suitable tertiary amines include triethanolamine. Although pyridine is not used as a solvent in the process of the present invention, a small amount of pyridine can be used as an acid scavenger. When used in small amounts, the pyridine can be purified out of the product with the other impurities.

The polar solvents and acid scavengers must be chemically compatible with the starting nucleosides, the reactants, and the nucleoside products to which they are exposed. They must also not interfere adversely with the reaction of the reactants with the nucleosides.

Because the DMT-Cl used in tritylation is highly reactive towards water, all materials involved in the tritylation must be anhydrous. In the preferred acylation processes of the present invention, Bz-dA may pick up water from the TMS-transient protection acylation reactions, and therefore need to be dehydrated. It was found that ordinary thermal and vacuum drying techniques were unsatisfactory for removing the water contained in this material, because the water may exist in hydrate form. In accordance with another aspect of the present invention, an azeotropic dehydration process has been developed which is effective to remove water in the Bz-dA.

The azeotropic dehydration process of the present invention is applied to the solution of the starting deoxyribonucleoside dissolved in the polar aprotic solvent prior to the addition of the protecting reagent. The dehydration process comprises adding a dehydrating solvent to the solution of step (a) that forms an azeotrope with water and the polar aprotic solvent, and distilling the azeotrope from the mixture. For a starting solution of deoxyribonucleoside in the polar aprotic solvent dimethylformamide (DMF) suitable dehydrating solvents include one or more C₅ to C₁₀ hydrocarbons, which may be linear, branched or cyclic, and may be substituted or unsubstituted. Preferred dehydrating solvents include pentane, hexane, toluene and heptane, particularly hexane. It was also found desirable to include a small amount of a tertiary amine, such as TEA, to stabilize the acylated nucleosides during the azeotropic dehydration step.

The protecting reagent should be added to the solution of the starting deoxyribonucleoside under controlled conditions of temperature and addition rate. The progress of the reaction should be monitored, as by HPLC analysis. The objective of the monitoring is to control and optimize the conversion without generating too much of over-tritylated impurities. The reaction is quenched by the addition of water when the optimal point is reached.

An additional advantage of tritylating the starting deoxyribonucleoside in the polar aprotic solvent rather than in pyridine is that the reaction does not require the use of a catalyst. As discussed above, tritylation in pyridine generally requires the use of a catalyst, such as 4-(dimethylamino)pyridine (DMAP) which is a toxic material that is very expensive and difficult to separate from the final product.

Step (c) is a purification step that removes polar impurities from the product. The solution of the protected nucleoside inevitably contains polar and non-polar impurities that must be removed from the final nucleoside product. As discussed above, the final product should be at least about 98% pure, preferably at least about 99% pure, and more preferably at least about 99.5% pure, by weight. Among the polar impurities, which need to be removed, are the amino-protected nucleosides which either did not react with the hydroxyl protecting agent, or which may have two acyl protecting groups (identified as bis-acylated products). Among the non-polar impurities, which need to be removed, are the bis-tritylated materials, which contain two trityl protecting groups, and impurities derived from DMT-Cl. These tend to be the most difficult impurities to remove because their polarity is relatively close to that of the desired protected nucleosides.

Polar impurities are removed from the solution of protected nucleoside product by a liquid-liquid extraction process, which takes advantage of the difference in solubility between protected nucleosides and polar impurities in polar solvent system. The protected nucleosides are generally insoluble in water and basic aqueous salt solutions, while many of the polar impurities are soluble in water or such solutions. Therefore water, or preferably an aqueous solution of water and a basic salt such as a soluble carbonate or bicarbonate salt, can be used to extract polar impurities from the protected nucleoside product. By adding water or a basic aqueous solution to the initial product solution in polar, aprotic solvent, the protected nucleoside is maintained to the non-polar phase, while the majority of polar impurities are left in the polar phase. Preferred basic salts include sodium and potassium carbonate and bicarbonate, particularly sodium bicarbonate. Preferably, the basic aqueous salt solution contains about 1% to about 10%, preferably about 2% to about 5%, by weight salt. Other halogenated solvents, such as chloroform and 1,2-dichloroethane may also be employed as the non-polar solvent. The product solution in the non-polar solvent can be repeatedly extracted in this manner with water or a basic aqueous solution to achieve higher purity level. It has been found that an aqueous solution comprising about 1 to about 10% by weight NaHCO₃, preferably about 2% to about 5%, and about 0 to about 40% DMF, is suitable for the removal of polar impurities. After the polar solvent system has been mixed with the non-polar phase, the mixture is allowed to settle, and is separated. The desired protected product remains in the non-polar phase, while the undesired polar impurities are carried away in the polar phase. The process may be repeated as many times as necessary to remove the polar impurities and obtain the desired product purity.

This polar solvent system selectively extracts out all or most of the polar impurities. To remove additional polar impurities, the resulting product solution optionally may be treated further with a suitable adsorbent, such as activated carbon. Other suitable adsorbents, such as silica, alumina, and molecular sieves, are well known to those skilled in the art. When polar impurities are below desirable levels, the adsorbent with adsorbed impurities may be readily removed, as by filtration. This has been found to be a desirable step in the purification of the protected dA products, but unnecessary in the purification of dG.

In step (d), the product is solidified out of solution by a process, which further purifies the product. The product may be solidified out of solution by crystallization, in the case of a crystalline product, by precipitation in the case of an amorphous product, or by a combination of crystallization and precipitation in the case of products, which form both crystalline and amorphous solids. Thus the term solidification is intended to encompass the processes of crystallization, precipitation and combinations thereof by which a solid product comes out of solution. Solidification may be effected by the use of either non-polar solvents or polar solvents. In non-polar solvent solidification, a non-polar phase containing the dissolved product is combined with a miscible solvent in which the product is insoluble in an amount effective to crystallize a crystalline product, or to precipitate an amorphous product from the non-polar phase. For crystallization, preferably, the miscible solvent in which the product is insoluble is added slowly to the product solution. For precipitation, preferably, the product solution is added slowly to the miscible solvent in which the product is insoluble.

When the solidification is taking place in a polar solvent, the product is maintained, transferred or re-dissolved into a polar phase, which comprises a polar solvent that is miscible with water. Because the present protected nucleosides are all insoluble in water, water can be used to solidify the product from the polar phase. To solidify the product from the polar phase, the polar phase containing the dissolved product may be combined with a suitable amount of water effective to solidify the product out of solution. The water may be added to the polar phase, or the polar phase may be added to the water.

Many polar solvents are suitable for use in this solidification process. In addition to being miscible with water and a solvent for the product, the polar solvent must also be inert to the product. Preferably, the polar solvent also has a boiling point of less than 100 degrees C., the boiling point of water, to facilitate subsequent removal by evaporation. Suitable solvents include acetonitrile, acetone, and lower (C₁ to C₃) alcohols, particularly methanol.

When the product is solidified in non-polar solvents, crystallization effectively removes most of the non-polar impurities. The non-polar impurities remain dissolved in the non-polar solvents as the product crystallizes out of solution. Such a crystallization process is preferably used with Bz-DMT-dA. However, when the product is solidified by precipitation to an amorphous form, it was found that additional purification might be needed to remove non-polar impurities. In such a case, an additional step of liquid-liquid extraction is used to remove non-polar impurities. Such a process is preferably used with protected nucleosides which form amorphous solids, as was found to be the case with iB-DMT-dG. In liquid-liquid extraction to remove non-polar impurities, the crude product is isolated in a polar phase in which it is soluble. A mixture of DMF and water is preferred for use in the liquid-liquid extraction purification of these products. The impurities are then extracted with a non-polar solvent, or a non-polar solvent system. The non-polar solvent should be a solvent for the non-polar impurities, but not for the nucleoside product. In addition, the polar phase and non-polar solvent system must be immiscible with each other. This will cause the non-polar impurities to partition into the non-polar solvent phase, while keeping the majority of the product in the polar solvent phase. The non-polar impurities can then be removed by phase separation. Mixtures of aromatic hydrocarbons and aliphatic hydrocarbons are suitable non-polar solvent systems, particularly ones containing about 6 to 12 carbon atoms, with or without heteroatoms. A mixture of cumene and hexane is suitable although a mixture of methyl t-butyl ether (MTBE) and toluene is a preferred composition. Preferably, the mixture comprises about 10 to about 90 parts aromatic to about 90 to about 10 parts aliphatic, by volume, more preferably about 1 to about 3 parts aromatic to 1 part aliphatic, with a mixture of about 1 parts aromatic to 1 part aliphatic being particularly preferred.

To solidify the product out of solution by precipitation or crystallization, the polar aprotic phase in which the product has been isolated, and from which the polar impurities have been removed is mixed with an immiscible non-polar solvent in which the product is insoluble. A wide range of solvents can be used for this purpose, so long as the non-polar phase and the non-polar solvent are miscible with each other. For crystalline products, the non-polar solvent is preferably added slowly to the non-polar phase comprising the product. This causes the product to crystallize, while keeping non-polar impurities in the mother liquor. For amorphous products, the non-polar phase comprising the product is preferably added slowly to the non-polar solvent. This causes the product to precipitate, while keeping the non-polar impurities in solution.

The solidification process is considered precipitation in the case of a product, which is amorphous in solid form, and crystallization in the case of a product, which is crystalline in solid form. By the use of this process, and repeating the process as needed, essentially pure protected nucleosides are obtained without the use of chromatography and without the use of pyridine as a solvent or DMAP as a catalyst. In addition, the process can be scaled up readily to any desirable scale. For purposes of this application, an essentially pure nucleoside is one of greater than about 98% purity, and preferably greater than about 99% purity, and most preferably greater than about 99.5% pure, all by weight.

The process of the present invention offers many advantages over prior processes in which pyridine is used as the process solvent. Because the polar aprotic solvent used in the present process are better solvents for the nucleosides than pyridine, the reactions can be run at much higher nucleoside concentrations. Thus, higher volumetric efficiency is obtained. As discussed above, the present process eliminates, or at least greatly reduces the use of pyridine and DMAP, which are considered highly toxic substances. The purification process steps of the present invention, including extraction, adsorption, and precipitation/crystallization are common operations, and are more efficient and less costly operations than chromatography. Because of the simplicity and other improvements of the present process, the overall product yield is also improved. Further, the combination of these improvements results in a significant lowering of the processing costs for protected nucleosides. Finally, the new processes are scalable to any desirable scale to meet industrial demand.

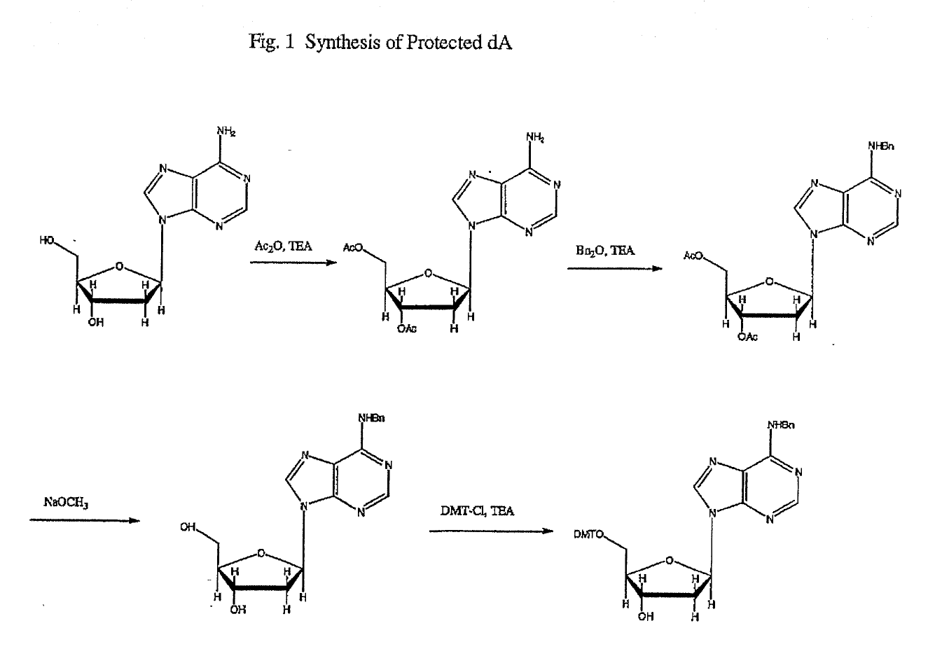

FIG. 1 presents a schematic representation of a preferred method of preparing protected N⁶-Benzoyl-5′-O-(4,4-dimethoxytrityl)-2′-deoxyadenosine from deoxyadenosine free-base, with the direct acylation protection. The deoxyadenosine free-base is combined with acetic anhydride to form transiently O-protected dA, followed by N-benzoylation. This material is then hydrolyzed to form the N-benzoyl compound which is tritylated by the addition of DMT-Cl to form the final protected product N⁶-Benzoyl-5′-O-(4,4-dimethoxytrityl)-2′-deoxyadenosine as shown in FIG. 1.

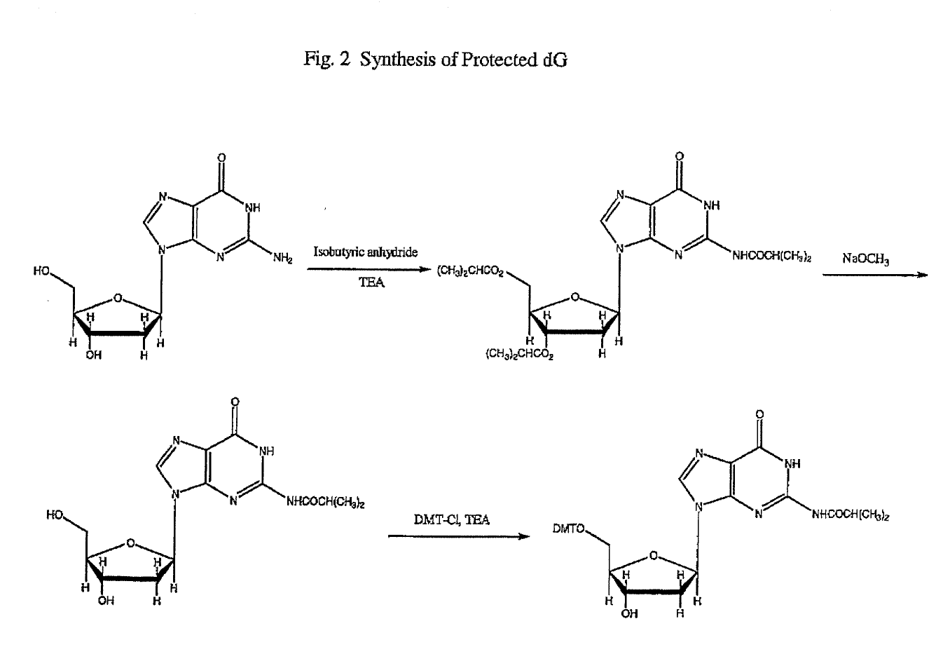

FIG. 2 presents a schematic representation of a preferred method for the preparation of protected 5′-O-dimethoxytrityl N²-isobutyryl-2′-deoxyguanosine from deoxyguanosine. In this case dG is tri-acylated with isobutyric anhydride followed by selective O-deprotection. This N-butyryl dG material is then tritylated by the addition of DMT-Cl to form the final protected product 5′-O-dimethoxytrityl N²-isobutyryl-2′-deoxyguanosine as shown in FIG. 2.

In the following examples, the solid protected form of dA is crystalline in structure, and therefore it is solidified out of solution by crystallization. On the other hand, dG is amorphous, and is therefore solidified by precipitation. Generally, the crystallization process successfully reduced the level of non-polar impurities to the desired level. Other particular steps in the, processing of the different nucleosides will be apparent from the following detailed descriptions of examples.

### EXAMPLES

In the following examples protected forms of the nucleosides deoxyadenosine (dA), and deoxyguanosine (dG) are made in accordance with the method of the present invention.

### Example 1

### Preparation of 5′-O-dimethoxytrityl N²-isobutyryl-2′-deoxyguanosine

Step (1a): N²-, 3′-O, 5′-O-tri-isobutyryl deoxyguanosine

A mixture of 200 g (0.70 mol) of deoxyguanosine (dG) monohydrate, 480 g of isobutyric anhydride (3.03 mol, 4.33 eq based on dG), 353 g of TEA (3.5 mol, 5.0 eq based on dG), and 1.0 L of DMF is heated to about 75-80° C. and stirred under nitrogen. The reaction is monitored using HPLC and samples taken every 30 minutes. After about 4 hr additional amounts of isobutyric anhydride is added to the reaction mixture to lower the dG and iB-dG level to below 3% (area %). When the combined dG and iB-dG level drops to less than 3%, the reaction is quenched with 1.51 of 5% NaHCO₃ solution. The quenched mixture is stirred for about 30 minutes, cooled to 25° C. and extracted with methylene chloride (3×0.5 L). The methylene chloride extracts are combined and washed with 5% NaHCO₃ solution (3×1 l), and the organic phase is separated and dehydrated by azeotropic distillation. The dehydrated organic phase is used as is in the next step.

Step (1b): N-isobutyryl deoxyguanosine

A chilled 25% methanolic solution of sodium methoxide (76 g, 0.35 mol. 0.5 eq based on dG) is added slowly to a cooled mixture (about −18 to about −20° C.) of the dehydrated organic phase from step (1a) above diluted in 2.0 l of methanol. The temperature of the mixture is maintained at or below −15° C. during the addition, and during the course of the de-protection reaction. Samples are taken about every 30 min. Additional NaOCH₃ is used to keep the reaction going and to lower the combined di- and triacylated dG level to below 3%. When the reaction is completed, 100 g of triethylammonium chloride (0.7 mol, 1 eq based on dG) is added to the mixture, which is stirred for 30 min at −15° C. and warmed up to 20° C. and stirred for an additional hour (at 20° C.). 2.0 l of DMF is added to the mixture, which is distilled at ambient pressure at about 80° C. to remove methanol, methylene chloride, methyl isobutyrate, triethylamine and other low-boiling components. When no more distillate is collected, 2.0 l of hexane is added to the mixture, which is distilled again to azeotropically remove any residual methanol.

Step (2): 5′-O-dimethoxytrityl N²-isobutyryl-2′-deoxyguanosine

1.5 l of a methylene chloride solution of 98% DMT-Cl (285 g; 0.84 mol, 1.20 eq based on initial dG) is added over a 30 min period to the mixture of triethylamine (141 g; 1.4 mol, 2.0 eq based on dG) and the organic residue from step (1b). The temperature of the mixture is controlled to about 25° C. during the addition after which the mixture is stirred for 30 min at 25° C. The mixture is analyzed by HPLC, and additional amounts of DMT-Cl (as solid) are added to lower the residual iB-dG level to below 3%. The mixture is washed with 3% NaHCO₃ solution (1×5.0 l), a mixture of DMF and 3% NaHCO₃ solution (1:2, 2×5.0 l), and 3% NaHCO₃ solution (2×5.0 l). The organic phase is analyzed by HPLC after each extraction and combined with a 50-50 mixture of MTBE and toluene (7 l) while vigorously stirring for about 30 min, further stirred for 30 min and filtered. The filter cake is dried under vacuum suction for 15 min and re-dissolved in 2 l of methylene chloride, re-precipitated with 7 L of 50-50 MTBE/toluene, and the filtering process repeated until the purity of the material is greater than 99.3%. The wet cake is dried in a vacuum oven until it passed the loss on drying (LOD). The typical yield is 75%.

### Example 2

### The Preparation of N⁶-Benzoyl-5′-O-(4,4-dimethoxytrityl)-2′-deoxyadenosine

Step (1a): 3′-O, 5′-O-diacetyl-2′-deoxyadenosine

A mixture of 2′-deoxyadenosine monohydrate (dA)(454 g), toluene (5 l), anhydrous triethylamine (TEA)(860 g), and of acetic anhydride (686 g) is stirred under nitrogen at 20° C. and sampled every 30 min until the combined residual dA and mono-acetylated dA (two isomers) is less than 2%. The mixture is cooled to 15° C. 3 l of 5% NaHCO₃ solution is added while maintaining the temperature below 25° C. The mixture is stirred at 25° C. for 30 min to hydrolyze the excess acetic anhydride. The mixture is allowed to settle, and the lower phase is removed. 3 l of 5% NaHCO₃ solution is added to the organic phases, the phases are mixed, and combined with 0.2 l of TEA and 2 l of hexane. The mixture is heated to 70° C. to strip off hexane and residual water. The process is continued until the water content is lower than 0.2% by Karl-Fisher titration. The O-acetylated product is used as is in the next step.

Step (1b): N6-Benzoyl-3′-O, 5′-O-diacetyl -2′-deoxyadenosine

The dried organic phase obtained in step (1a) is combined with TEA (330 g) and benzoic anhydride (Bn₂O) (570 g), stirred under nitrogen at 85° C., sampled on an hourly basis until benzoylation is completed (<2% residual 2 Ac-dA), and cooled to 45° C. A 5% NaHCO₃ solution (4 l) is added to the reaction mixture while keeping the temperature below 45° C. Upon the completion of the addition, the mixture is stirred at 45° C. for 1 hr to hydrolyze the residual Bn₂O, and allowed to phase separate. The organic phase is separated and washed with 1% NaHCO₃ solution (2×5.0 L) to remove all water-soluble components.

Step (1c): N⁶-Benzoyl-2′-deoxyadenosine.

A methanolic NaOCH₃ solution (25%; 362 g) is added to a cooled mixture of the washed organic phase obtained in step (1b) and methanol (2 l) while maintaining the temperature below −13° C. Upon completion of the addition, the mixture is stirred at −15° C., and sampled every 30 minutes until the reaction is complete and is supplemented with additional NaOCH₃ solution as necessary. The reaction is quenched with 4 l of 10% TEA-HCL solution, is stirred at 0° C. for 20 min, and the temperature raised to 70° C. When all solids dissolve, the mixture is allowed to settle, and the aqueous phase removed. The organic phase is washed with 3 l of 1% NaHCO₃ solution at 70° C., the aqueous phase removed and the organic phase distilled to remove 3 l of toluene phase under reduced pressure at 70° C. The residue is diluted with TEA (450 g), DMF (3 l), and cooled to 20° C., and used as is in the next step.

Step (2): N⁶-Benzoyl-5′-O-(4,4-dimethoxytrityl)-2′-deoxyadenosine

A methylene chloride solution of DMT-Cl (683 g in 3 l of CH₂CL₂) is added to the diluted organic phase obtained in step (1c) while maintaining the mixture temperature below 25° C. Upon the completion of the addition, the mixture is stirred at 25° C. and sampled every 30 min until the residual Bz-dA is less than 5% (area). Suitable amounts of additional DMT-Cl are added to the mixture as necessary. When the desired conversion is achieved, the mixture is quenched with 4 l of 5% NaHCO₃ solution. The organic phase is washed twice with a mixture of DMF and 5% NaHCO₃ solution (1:3, 4 l each time), and then again with a 5% NaHCO₃ solution (4 l). The washed organic is combined gradually with 3 l of MTBE-hexane mixture (1:1) with vigorous agitation. Upon the completion of the addition, the mixture is cooled to 5° C. and stirred at mild agitation rate to allow nucleation to take place. When the mixture turns into a slurry, 2 l of hexane are added, and the resulting mixture stirred at 5° C. for 2 hr. The solid is filtered, washed with 4 l of MTBE-hexane mixture (1:1), and vacuum dried until the residual solvent level is lower than 15% (by GC). The solid is re-dissolved in 2 l of CH₂Cl₂, and the product re-precipitated by slowly adding 7 l of MTBE-hexane mixture. The mixture is cooled to 5° C., and the solid was filtered. The filtering step is repeated until the purity is greater than 99.0%. The material is dried in a vacuum oven at 50° C. until it passes LOD. The overall yield is 65%.

We claim:

1. A process for the preparation of an N-acyl deoxynucleoside, which is either an N-acyl deoxyadenosine or an N-acyl deoxyguanosine, comprising acylating the hydroxyl groups and the exocyclic amino group on said deoxynucleoside with anhydride to form a 3′-, 5′O-acyl, N-acyl deoxynucleoside and selectively removing the acyl groups from the hydroxyl groups to form an N-acyl deoxyadenosine or N-acyl deoxyguanosine.

2. The process according to claim 1 wherein said anhydride comprises an alkyl anhydride, or an alkyl anhydride and an aryl anhydride.

3. The process according to claim 2 wherein said alkyl anhydride is isobutyric anhydride or acetic anhydride.

4. The process according to claim 1 wherein said deoxynucleoside is deoxyguanosine.

5. The process according to claim 1 wherein said acyl groups are removed under conditions using a nucleophilic reagent.

6. The process according to claim 5 wherein said conditions comprise anhydrous alkoxide.

7. The process according to claim 5 wherein said conditions comprise anhydrous sodium methoxide and a temperature range from about −25° C. to about −10° C.

8. A process for the preparation of an N-acyl derivative of a deoxynucleoside containing 3′- and 5′-hydroxyl groups and an exocyclic amino group, comprising the steps of:
(1) reacting said deoxynucleoside with a first anhydride under conditions effective in selectively acylating said hydroxyl groups;
(2) reacting the 3′-, 5′-O-acylated product of step (1) with a second anhydride under conditions effective in acylating said primary amino group to form an N-acylated, 3′-, 5′O-acylated deoxynucleoside; and
(3) subjecting said N-acylated, O-acylated deoxynucleoside to conditions effective to selectively remove said O-acyl groups to form N-acyl deoxynucleoside, wherein said deoxynucleoside is either deoxyadenosine or deoxyguanosine.

9. The process according to claim 8 wherein the deoxynucleoside is deoxyadenosine.

10. The process according to claim 9 wherein said first anhydride is isobutyric anhydride or acetic anhydride.

11. The process according to claim 10 wherein said second anhydride is benzoic anhydride.

12. The process according to claim 11 wherein said O-acyl groups are selectively removed under anhydrous nucleophilic conditions.

13. The process according to claim 12 wherein said conditions comprise the use of an anhydrous alkoxide within a temperature range from about −25° C. to about −10° C.

14. In a process for the protection of the exocyclic amino group and 5′-hydroxyl group contained in either deoxyadenosine or deoxyguanosine, comprising the transient protection of the 3′- and 5′-hydroxyl groups, the acylation of said amino group, the selective removal of said hydroxyl group protection and a non-transient protection of said 5′-hydroxyl group, wherein the improvement comprises the selective transient acylation of said deoxynucleoside hydroxyl groups, the acylation of the exocyclic amino group, and the selective removal of the O-acyl protecting groups.
